(19) United States
(12) Patent Application Publication
(10) Pub. No.: US 2017/0246619 A1
(43) Pub. Date: Aug. 31, 2017

(54) SOLID STATE SYNTHESIS OF OXIDATIVE DEHYDROGENATION CATALYSTS

(71) Applicant: Dow Global Technologies LLC, Midland, MI (US)

(72) Inventors: Jean-Marie Basset, Thuwal (SA); Haibo Zhu, Thuwal (SA); Devon C. Rosenfeld, Freeport, TX (US); Paco Laveille, Thuwal (SA)

(73) Assignee: Dow Global Technologies LLC, Midland, MI (US)

(21) Appl. No.: 15/508,523

(22) PCT Filed: Sep. 18, 2015
(86) PCT No.: PCT/US15/50840

§ 371 Date: Mar. 3, 2017

Related U.S. Application Data
(60) Provisional application No. 62/056,132, filed on Sep. 26, 2014.

(51) Int. Cl. B01J 23/888; B01J 23/847; C07C 5/48; B01J 37/04; B01J 37/08; B01J 23/755; B01J 23/835

(52) U.S. Cl. CPC B01J 23/888; B01J 23/755; B01J 23/8476; B01J 23/8474; B01J 23/835; B01J 37/04; B01J 37/088; C07C 5/48; C07C 2523/755; C07C 2523/847; C07C 2523/888; C07C 2523/835

(57) ABSTRACT

Synthesize a nickel oxide-based oxidative dehydrogenation catalyst via a solvent-free process that comprises sequential steps a. mixing without added solvent a combination of a solid nickel precursor, a solid oxalate or oxalic acid and, optionally, a doping amount of a metal precursor for a period of time sufficient to convert the combination to a visually homogenous mixture; and b. calcining the visually homogeneous mixture at a temperature within a range of from greater than 250° C. to less than 800° C. for a time within a range of from 30 minutes to 360 minutes in an oxygen-containing atmosphere, preferably air, to form a calcined oxidative dehydrogenation catalyst. As a modification of the process, add an intermediate step between steps a. and b. to dry the homo geneous mixture at a temperature within a range of from 50° C. to 90° C. for a period of time within a range of from 10 minutes to 600 minutes to form a dried mixture. The resulting catalyst may be used in oxidative dehydrogenation of ethane.

The present application claims the benefit of U.S. Provisional Application No. 62/056,132, filed on Sep. 26, 2014.

This invention relates generally to synthesis and use of oxidative dehydrogenation catalysts, especially to solid state synthesis of such catalysts and use of such catalysts in oxidative dehydrogenation of ethane. This invention relates more particularly to such catalysts that are nickel oxide (NiO)-based catalysts.

Ethylene is a key raw material for synthesis of a wide variety of products including polymers, fine chemicals, plastics, and fibers. Currently, ethylene production involves steam cracking of a hydrocarbon feedstock, such as naphtha or ethane, at a relatively high temperature (e.g. 750 degrees centigrade (° C.) to 900° C.). As such, many regard it as one of the most energy-consuming processes in the chemical industry. It reportedly results in a global use of approximately eight percent (8%) of the sector's total primary energy use, excluding energy content of final products.

Ethylene produced by oxidative dehydrogenation (ODH) process at relatively, in comparison to steam cracking, low temperature (e.g. from 300° C. to 500° C.) is a potentially attractive alternative route compared with the traditional steam cracking route. In the ethane ODH process, no additional heat is required to sustain the reaction, because ODH is an exothermic reaction. Also, catalyst deactivation from coke formation is suppressed due to the presence of oxygen.

NiO is known to be very reactive and capable of activating ethane at moderate temperature (below 400° C.). Furthermore, the physical and chemical properties of NiO can be modified and improved by doping with transition metals, such as niobium (Nb), zirconium (Zr), tungsten (W), and tin (Sn) or by supporting it on a carrier such as silica (SiO₂), alumina (Al₂O₃), zirconia (ZrO₂), magnesia (MgO) or ceria (CeO₂). Literature reports that the resulting NiO based materials deliver increased ethylene yield. See, e.g., several references by Yumin Liu et al. including U.S. Pat. No. 7,498,289B2, U.S. Pat. No. 7,227,049B2, U.S. Pat. No. 6,335,854, U.S. Pat. No. 6,436,871, U.S. Pat. No. 6,677,497, U.S. Pat. No. 6,777,371, U.S. Pat. No. 6,891,075, U.S. Pat. No. 6,891,075, U.S. Pat. No. 7,674,944 and U.S. Pat. No. 6417422B1. A common thread through such references is preparation of Ni-containing catalysts from an aqueous solution via a precipitation process.

Related cases U.S. Pat. No. 7,498,289, U.S. Pat. No. 7,626,068 and U.S. Pat. No. 7,674,944 all teach catalysts and methods for alkane (e.g. a two to six carbon atom (C₂-C₆) alkane such as ethane or propane) oxydehydrogenation. The catalysts comprise (i) nickel or a nickel-containing compound and at least one of (ii) titanium (Ti), tantalum (Ta), Nb, hafnium (Hf), W, yttrium (Y), zinc (Zn), Zr or aluminium (Al) or a compound containing at least one of such elements. General approaches for preparing nickel catalysts include sol-gel, freeze drying, spray drying, precipitation, impregnation, incipient wetness, spray impregnation, ion exchange, wet mix/evaporation, dry mix/compacting, high coating, fluid bed coating, bead coating, spin coating, physical vapour deposition (sputtering, electron beam evaporation, laser ablation) and chemical vapour deposition. See also Yumin Liu et al., “Discovery from combinatorial heterogeneous catalysis: A new class of catalyst for ethane oxidative dehydrogenation at low temperatures”, Applied Catalysis A: General 254 (2003) 59-66 which focuses on catalyst preparation via the sol-gel method or the evaporation method. None of the techniques so disclosed use oxalic acid or an oxalate precursor along with Ni and a transition metal or doping metal precursor in a solvent-free process.

B. Solsona et al, in “Selective oxidative dehydrogenation of ethane over SnO₂-promoted NiO catalysts”, Journal of Catalysis 295 (2012) 104-114, discloses preparation of the title catalysts via evaporation at 60° C. of a stirred ethanolic solution of nickel nitrate hexahydrate and tin oxalate (SnC₂O₄) followed by drying overnight at 120° C. and then calcination in air for two hours at 500° C. Oxalic acid is added to the solution with a molar ratio of oxalic acid to the sum of nickel and tin of 1 for consistency.

Haibo Zhu et al., in “Nb effect in the nickel oxide-catalyzed low-temperature oxidative dehydrogenation of ethane”, Journal of Catalysis 285 (2012), 292-303, teaches a method for preparing NiO and Nb/NiO nanocomposites based on slow oxidation of a nickel-rich Nb Ni gel obtained in citric acid. In one protocol, Zhu et al. prepares nickel oxides via precipitation by reaction between nickel nitrate and oxalic acid in aqueous solution.

E. Heracleous et al., in “Ni—Nb—O mixed oxides as highly active and selective catalysts for ethene production via ethane oxidative dehydrogenation. Part I: Characterization and catalytic performance”, Journal of Catalysis 237 (2006) 162-174, relates to bulk Ni—Nb—O mixed oxides and their preparation via evaporation using aqueous solutions of precursor salts nickel nitrate hexahydrate and ammonium niobium oxalate. See also E. Heracleous et al., in “Ni—Nb—O mixed oxides as highly active and selective catalysts for ethene production via ethane oxidative dehydrogenation. Part II: Mechanistic aspects and kinetic modeling”, Journal of Catalysis 237 (2006) 175-189. See also Z. Skoufa et al., “Unraveling the contribution of structural phases in Ni—Nb—O mixed oxides in ethane oxidative dehydrogenation”, Catalysis Today 192 (2012) 169-176, and Z. Skoufa et al., “Investigation of engineering aspects in ethane ODH over highly selective Ni_{0.85}Nb_{0.15}Ox catalyst” Chemical Engineering Science 84 (2012) 48-56.

B. Sasova et al., in “Ni—Nb—O catalysts for ethane oxidative dehydrogenation”, Applied Catalysis A: General 390 (2010) 148-157, relates to the title catalysts with various Nb contents that range from 0 wt % to 19 wt % that are prepared with ammonium oxalate niobate as a niobium precursor via an aqueous evaporation method.

A desire exists for a liquid-free procedure to synthesize NiO-based low temperature (250° C. to 350° C.) ethane ODH catalysts. Such a procedure offers several advantages including classification as a relative low cost, compared to liquid synthesis, green procedure (no added water or solvent use resulting in no contaminated water or solvent).

In some aspects, this invention is a solvent-free process for synthesizing a nickel oxide-based oxidative dehydrogenation catalyst that comprises sequential steps as follows:

a. mixing without added solvent a combination of a solid nickel precursor, a solid oxalate or oxalic acid and, optionally, a doping amount of a metal precursor for a period of time sufficient to convert the combination to a visually homogenous mixture; and

b. calcining the visually homogeneous mixture at a temperature within a range of from greater than 250° C. to less than 800° C. for a time within a range of from 30 minutes to 360 minutes in an oxygen-containing atmosphere, preferably air, to form a calcined oxidative dehydrogenation catalyst.

In some aspects, the solvent-free process further comprises a sequential intermediate step a′ that follows step a, precedes step b and comprises drying the homogeneous mixture at a temperature within a range of from 50° C. to 90° C. for a period of time within a range of from 10 minutes to 600 minutes to form a dried mixture, the dried mixture thereby replacing the visually homogeneous mixture in step a.

The foregoing catalyst preparation process variations have utility in simplifying catalyst preparation and providing a catalyst that, as demonstrated in examples and comparative examples shown below, has improved performance in oxidative dehydrogenation of ethane when compared to a catalyst having the same composition that is prepared via sol-gel synthesis.

As used herein, “dry mixing” and “solvent-free” both refer to mixing in the absence of an added solvent, whether aqueous or organic.

In related aspects, this invention is a process for effecting oxidative dehydrogenation of ethane using the above nickel oxide-based oxidative dehydrogenation catalyst comprising sequential steps as follows:

a. placing the calcined oxidative dehydrogenation catalyst in contact with a feedstream that comprises ethane, an oxygen-containing gas such as air, enriched air or oxygen and, optionally, an inert diluent selected from helium (He), nitrogen (N₂) and argon (Ar), at a temperature of from greater than 200° C. to less than 400° C., at a pressure of from one atmosphere to 20 bars (2×10⁶ pascals), a feedstream flow rate within a range of from 50 hr⁻¹ to 10000 hr⁻¹ and a feedstream molar ratio of molecular oxygen to ethane within a range of from 0.01:1 to 1:1 to yield a product stream that comprises ethylene, carbon dioxide and unreacted ethane.

b. recovering ethylene from the product stream.

In other aspects, this invention offers a simple but general and robust method for synthesizing NiO based materials. A wide range of transition metals can be incorporated into the NiO matrix, forming highly active catalysts for ethane ODH.

Catalyst synthesis in accord with this invention begins by physically mixing a combination of solid nickel precursor, a solid oxalate salt or oxalic acid and, optionally, a doping amount of a transition metal precursor in the absence of a solvent (e.g. water, a water solution or an organic solvent), for a period of time sufficient to convert the mixture of individual catalyst components to an intimate mixture. Physical mixing may occur in any of a variety of physical mixing apparatus including, without limit, a mortar and pestle, a lidded container, the contents of which may be shaken, a ball mill, a blender, a grinder or a stirred pot. The period of time varies with the apparatus with suitable times ranging from 5 minutes to 120 minutes, preferably from 5 minutes to 60 minutes for a mortar and pestle and from 2 minutes to 40 minutes for a blender or grinder. The intimate mixture has, relative to the mixture, a smaller average particle size.

The solid nickel precursor, the oxalate and, when used, the transition metal are present in amounts as follows: from 1 percent by mole (mol %) to 40 mol %, preferably from 3 mol % to 30 mol %, and more preferably from 5 mol % to 20 mol %, solid nickel precursor; from greater than 20 mol % to 98 mol %, preferably from 40 mol % to 94 mol %, and more preferably from 60 mol % to 90 mol %, oxalate and from greater than or equal to lmol % to 40 mol %, preferably from 3 mol % to 30 mol %, and more preferably from 5 mol % to 20 mol %, transition metal, each mol % being based upon combined moles of nickel precursor, oxalate and transition metal and, in each case, when added together total 100 mol %.

The solid nickel precursor is selected from a group consisting of nickel nitrate, nickel hydroxide, nickel acetate and their corresponding hydrated compounds.

The oxalate is selected from a group consisting of oxalic acid, ammonium oxalate, sodium oxalate, potassium oxalate monohydrate, preferably oxalic acid or ammonium oxalate.

The dopant metal precursor is selected from compounds of Groups IV through VI of the Periodic Table of the Elements, iron (Fe) and tin (Sn), preferably from compounds of a group consisting of tantalum (Ta), niobium (Nb), titanium (Ti), molybdenum (Mo), tungsten (W) and zirconium (Zr).

When the metal oxalate salts, such as niobium oxalate, tin oxalate, containing both oxalate and dopant metal are used as the precursor, no additional oxalate precursor is required for the synthesis.

Nickel precursors, oxalates and dopant metal precursors may contain bound water. In the process of this invention, a preferred embodiment is to remove such water, e.g. by drying at a temperature within a range of from 50° C. to 90° C. for a period of time within a range of from 10 minutes to 600 minutes to form a dried powder.

The catalyst synthesis process of this invention continues by calcining the intimate mixture at a temperature of from greater than 250° C. to less than 800° C. for a time within a range of from 30 minutes (min) to 360 min, preferably from 120 min to 240 min in an oxygen-containing atmosphere, preferably air, to form a calcined oxidative dehydrogenation catalyst.

The resulting calcined catalysts are crystalline materials that exhibit a cubic rock salt structure typical of a NiO crystal. The transition metal is homogeneously incorporated into the lattice of NiO crystal. Surface areas of the catalysts vary between 20 square meters per gram (m²/g) and 180 m²/g depending on both calcination temperature and doping metal content. The size of NiO crystallites lies within a range of from 5 nm and 25 nm, and it is essentially affected by the ratio of transition metal.

### EXAMPLE (Ex) 1

### Synthesis of NiO_{—} Catalyst

Place 4.74 grams (g) of nickel nitrate hexahydrate (Ni(NO₃)₂.6H₂O) and 1.13 g of oxalic acid (H₂C₂O₄) in a mortar bowl. Using a pestle, mix and grind the mortar bowl contents at room temperature (nominally 25° C.) for 10 minutes to get a uniform paste. Dry the paste at 90° C. for 2 hours. Calcine the dried paste under static air at 300° C. for 4 hours to produce a black solid.

### EX 2

### Synthesis of Ni_{0.99}Ta_{0.01}O Catalyst

Replicate Ex 1 but add 0.067 grams (g) of tantalum (V) ethoxide (Ta(OCH₂CH₃)₅) to the mixture of Ex 1.

### EX 3

### Synthesis of Ni_{0. 97}Ta_{0.03}O Catalyst

Replicate Ex 1 but add 0.21 g of tantalum(V) ethoxide (Ta(OCH₂CH₃)₅) to the mixture of Ex 1.

### EX 4

### Synthesis of Ni_{0.95}Ta_{0.05}O Catalyst

Replicate Ex 1 but add 0.35 g of tantalum(V) ethoxide (Ta(OCH₂CH₃)₅) to the mixture of Ex 1.

### EX 5

### Synthesis of Ni_{0.93}Ta_{0.07}O Catalyst

Replicate Ex 1 but add 0.50 g of tantalum(V) ethoxide (Ta(OCH₂CH₃)₅) to the mixture of Ex 1.

### EX 6

### Synthesis of Ni_{0.95}Nb_{0.05}O Catalyst

Replicate Ex 1 but change the amount of Ni(NO₃)₂.6H₂O to 1.98 g and substitute 0.24 g of niobium (V) oxalate hydrate (C₁₀H₅NbO₂₀.xH₂O) for the oxalic acid H₂C₂O₄.

### EX 7

### Synthesis of Ni_{0.95}W_{0.05}O Catalyst

Replicate Ex 1 but add 8.20 g of tungsten(VI) ethoxide (W(OCH₂CH₃)₆, 5% w/v in ethanol) into the mixture of Ni(NO₃)₂.6H₂O and H₂C₂O₄. Evaporate the ethanol from the tungsten(VI) ethoxide before adding it to the mixture.

### EX 8

### Synthesis of Ni_{0.95}Ti_{0.05}O Catalyst

Replicate Ex 1 but add 0.20 g of titanium ethoxide (Ti(OCH₂CH₃)₄) into the mixture of Ni(NO₃)₂.6H₂O and H₂C₂O₄.

### EX 9

### Synthesis of Ni_{0.95}Zr_{0.05}O Catalyst

Replicate Ex 1 but add 0.42 g of zirconium acetylacetonate (Zr(C₅H₇O₂)₄) into the mixture of Ni(NO₃)₂.6H₂O and H₂C₂O₄.

### EX 10

### Synthesis of Ni_{0.85}Nb_{0.15}O Catalyst

Replicate Ex 6 but change the amount of niobium (V) oxatate hydrate C₁₀H₅NbO₂₀.xH₂O to 0.80 g.

### EX 11

### Synthesis of Ni_{0.90}Ta_{0.10}O Catalyst

Replicate Ex 2 with modifications to change the amount of tantalum (V) ethoxide (Ta(OCH₂CH₃)₅) to 0.74 g.

### EX 12

### Synthesis of Ni_{0.85}Ta_{0.15}O Catalyst

Replicate Ex 2 with modifications to change the amount of tantalum(V) ethoxide (Ta(OCH₂CH₃)₅) to 1.17 g.

### EX 13

### Synthesis of Ni_{0.80}Ta_{0.20}O Catalyst Replicate Ex 2 with modifications to change the amount of tantalum(V) ethoxide (Ta(OCH₂CH₃)₅) to 1.66 g.

### EX 14

### Synthesis of Ni_{0.90}Nb_{0.10}O Catalyst

Replicate Ex 6 but change the amount of niobium (V) oxalate hydrate C₁₀H₅NbO₂₀.xH₂O to 0.51 g.

### EX 15

### Synthesis of Ni_{0.80}Nb_{0.20}O Catalyst

Replicate Ex 6 but change the amount of niobium (V) oxalate hydrate C₁₀H₅NbO₂₀.xH₂O to 1.14 g.

### EX 16

### Synthesis of Ni_{0.95}Sn_{0.05}O Catalyst

Replicate Ex 9 but substitute 0.30 g of tin(IV) acetate (Sn(CH₃CO₂)₄) for the zirconium acetylacetonate.

### EX 17

### Solid-State Synthesis of Ni_{0.85}Nb_{0.15}O with Nickel Hydroxide (Ni(OH) as Ni Precursor

Replicate Ex 10 but substitute 0.63 g of Ni(OH)₂for Ni(NO₃)₂.6H₂O.

### EX 18

### Solid-State Synthesis of Ni_{0.85}Nb_{0.15}O with Nickel Acetate (Ni(CH₃CO₂)₂) as Ni Precursor

Replicate Ex 10 but substitute 1.20 g of Ni(CH₃CO₂)₂ for Ni(NO₃)₂.6H₂O.

### EX 19

### Solid-State Synthesis of Ni_{0.85}Nb_{0.15}O Without Dry Process

Replicate Ex 10 but calcine the mixture after grinding directly without drying process a′.

### Comparative Example (CEx) A

### Synthesis of Pure NiO Without Oxalic Acid

Dry 2 g of Ni(NO₃)₂.6H₂O at 90° C. for 2 hours. Calcine the dried paste under static air at 300° C. for 4 hours to produce a black solid.

CEx B-F-sol-gel Synthesis of Ni₁₋ₓTaₓO (x=0.00, 0.01, 0.03, 0.05, and 0.07) Catalysts

In a glass vessel, dissolve six (6) g of nickel nitrate hexahydrate (Ni(NO₃)₂.6H₂O) and an amount of tantalum tetraethoxyacetylacetonate (Ta(OC₂H₅)₄(C₅H₇O₂)), 0.00 g (for Ni0 (CEx B), 0.10 g (for Ni_{0.99}Ta_{0.01}O) (CEx C), 0.29 g (for Ni_{0.97}Ta_{0.03}O) (CEx D), 0.51 g (for Ni_{0.95}Ta_{0.05}O) (CEx E) or 0.71 g (for Ni_{0.93}Ta_{0.07}O) (CEx F) in 100 mL of water to form a solution. Add two (2) g of citric acid to the solution which then turns blue. Age the blue solution at 80° C. for 24 hr. Evaporate water from the aged solution at 90° C. to form a gel. Dry the gel at 120° C. for 2 hr, 140° C. for 2 hr, and 160° C. for 12 hr to yield a black xerogel. Calcine the xerogel at 450° C. (ramp rate of 1° C./min from room temperature (nominally 25° C.) to 450° C.) for 4 hr in static air.

CEx G-sol-gel Synthesis of Ni_{0.95}Nb_{0.05}O

Replicate CEx C but substitute 0.35 g of niobium ethoxide (Nb(OCH₂CH₃)₄) for the tantalum tetraethoxyacetylacetonate.

CEx H-sol-gel Synthesis of Ni_{0.95}W_{0.05}O

Replicate CEx G but substitute 0.28 g of tungstic acid (H₂WO₄) for the niobium ethoxide.

CEx I-sol-gel Synthesis of Ni_{0.95}Ti_{0.05}O

Replicate CEx H but substitute 0.25 g of titanium ethoxide (Ti(OCH₂CH₃)₄) for the tungstic acid.

CEx J-sol-gel Synthesis of Ni_{0.95}Zr_{0.05}O

Replicate CEx I but substitute 0.42 g of zirconium(IV) butoxide Zr(OBu)₄ for the titanium ethoxide.

CEx K-solid-state Synthesis of Ni_{0.85}Nb_{0.15}O Without Oxalate

Replicate Ex 10 but substitute 0.38 g of niobium (V) ethoxide (Nb(OCH₂CH₃)₅)) for niobium (V) oxalate hydrate C₁₀H₅NbO₂₀.xH₂O.

CEx L—Solid-State Synthesis of Ni_{0.85}Nb_{0.15}O with Nickel Dichloride (NiCl₂)as Ni Precursor

Replicate Ex 10 but substitute 0.88 g of NiCl₂ for Ni(NO₃)₂.6H₂O. The resulting catalyst shows no activity and, as such, is not reported in Table 1.

### Catalyst Activity Evaluation

Evaluate catalytic activity of the NiO catalysts of Ex 1 to 19 and CEx A to L for oxidation of ethane to ethylene using a P&ID micro-pilot apparatus equipped with a stainless steel reactor (internal diameter 4 millimeters (mm)) at atmospheric pressure (nominally 14.5 pounds per square inch (psi) (1.013250×10⁵ pascals (Pa)). Load 100 milligrams (mg) of the catalyst into the reactor with glass wool as a support to form a catalyst bed that has a height of approximately 5 mm. Pass a feedstream composed of 10% C₂H₆/5% O₂ in He through the catalytic bed at a constant flow rate of 600 ml reciprocal hours (hr⁻¹). Heat the catalytic bed up to a temperature within a range of from 250° C. to 350° C. at a heating rate of 1° C. min⁻¹ to carry out the catalytic test at as shown in Table 1 below. Sample the reaction mixture at the outlet of the reactor at regular intervals, typically every 5 min, and analyze the reaction mixture using an on-line Varian 490 micro-GC equipped with a TCD (Thermal Conductivity Detector) and two columns, a MolSieve™ 5 Å column (Ar as carrier gas) to quantify O₂, and a poraPLOT Q™ column (He as carrier gas) to quantify CO₂, C₂H₄ and C₂H₆. Calculate ethane conversion and selectivity to ethylene on a carbon basis.

TABLE 1

| | | 300° C. | | | 330° C. | | | 350° C. | | |
|---|---|---|---|---|---|---|---|---|---|---|
| Ex/ | | % | % | % | % | % | % | % | % | % |
| CEx | Catalyst | Conv | Sel | Yld | Conv | Sel | Yld | Conv | Sel | Yld |
| 1 | NiO | 16.2 | 64.5 | 10.4 | 27.7 | 64.4 | 17.8 | 30.7 | 64.3 | 19.7 |
| A | NiO | 14.1 | 43.8 | 6.1 | 20.3 | 48.3 | 9.8 | 20.5 | 49.0 | 10.0 |
| B | NiO | 7.62 | 66.1 | 5.0 | 16.1 | 65.1 | 10.5 | 22.7 | 64.5 | 14.6 |
| 2 | Ni_{0.99}Ta_{0.01}O | 19.0 | 73.1 | 13.8 | 32.4 | 72.1 | 23.4 | 36.0 | 71.6 | 25.8 |
| C | Ni_{0.99}Ta_{0.01}O | 8.8 | 73.8 | 6.5 | 18.1 | 72.0 | 13.0 | 25.6 | 70.8 | 18.1 |
| 3 | Ni_{0.97}Ta_{0.03}O | 19.0 | 79.8 | 15.1 | 33.7 | 77.5 | 26.1 | 39.4 | 76.2 | 30.2 |
| D | Ni_{0.97}Ta_{0.03}O | 5.9 | 84.7 | 5.0 | 13.2 | 82.0 | 10.8 | 20.2 | 80.0 | 16.2 |
| 4 | Ni_{0.95}Ta_{0.05}O | 16.2 | 83.5 | 15.0 | 30.0 | 81.2 | 28.5 | 39.1 | 79.4 | 37.3 |
| E | Ni_{0.95}Ta_{0.05}O | 5.2 | 87.7 | 4.6 | 11.8 | 85.0 | 10.0 | 18.6 | 82.7 | 15.4 |
| 5 | Ni_{0.93}Ta_{0.07}O | 18.0 | 85.6 | 15.4 | 32.6 | 82.0 | 26.7 | 40.5 | 79.5 | 32.2 |
| F | Ni_{0.93}Ta_{0.07}O | 2.3 | 88.0 | 2.0 | 5.7 | 88.0 | 5.0 | 9.7 | 86.0 | 8.3 |
| 6 | Ni_{0.95}Nb_{0.05}O | 9.9 | 62.7 | 6.2 | 20.3 | 64.4 | 13.1 | 28.1 | 66.2 | 18.6 |
| G | Ni_{0.95}Nb_{0.05}O | 6.6 | 82.0 | 5.4 | 14.0 | 79.2 | 11.1 | 22.0 | 77.0 | 16.9 |
| 7 | Ni_{0.95}W_{0.05}O | 10.2 | 79.2 | 8.1 | 20.2 | 78.2 | 15.8 | 26.0 | 77.5 | 20.2 |
| H | Ni_{0.95}W_{0.05}O | 2.4 | 83.5 | 2.0 | 6.0 | 80.3 | 4.8 | 10.1 | 78.5 | 7.9 |
| 8 | Ni_{0.95}Ti_{0.05}O | 21.0 | 68.2 | 14.3 | 29.4 | 68.2 | 20.1 | 29.2 | 68.0 | 19.9 |
| I | Ni_{0.95}Ti_{0.05}O | 4.8 | 79.2 | 3.8 | 11.1 | 79.1 | 8.8 | 17.4 | 76.3 | 13.3 |
| 9 | Ni_{0.95}Zr_{0.05}O | 19.6 | 58.0 | 11.4 | 23.8 | 57.8 | 13.8 | 23.8 | 57.8 | 13.8 |
| J | Ni_{0.95}Zr_{0.05}O | 6.8 | 66.6 | 4.5 | 14.6 | 66.1 | 9.7 | 21.6 | 65.7 | 14.2 |
| 10 | Ni_{0.85}Nb_{0.15}O | 16.3 | 82.4 | 13.4 | 30.2 | 80.0 | 24.2 | 38.3 | 78.1 | 29.9 |
| K | Ni_{0.85}Nb_{0.15}O | 13.5 | 53.6 | 7.2 | 22.4 | 56.8 | 12.7 | 57.2 | 23.6 | 13.5 |
| 11 | Ni_{0.90}Ta_{0.10}O | 15.6 | 83.9 | 14.9 | 29.2 | 81.5 | 27.9 | 37.8 | 80.0 | 36.4 |
| 12 | Ni_{0.85}Ta_{0.15}O | 12.6 | 87.8 | 12.8 | 24.8 | 84.6 | 24.4 | 34.1 | 82.0 | 32.9 |
| 13 | Ni_{0.80}Ta_{0.20}O | 12.0 | 89.4 | 13.2 | 24.1 | 86.3 | 25.0 | 33.5 | 83.3 | 33.3 |
| 14 | Ni_{0.90}Nb_{0.10}O | 12.1 | 78.9 | 9.5 | 24.4 | 78.1 | 19.1 | 33.5 | 77.5 | 26.0 |
| 15 | Ni_{0.80}Nb_{0.20}O | 14.1 | 79.8 | 11.3 | 27.1 | 77.8 | 21.1 | 35.7 | 76.5 | 27.3 |
| 16 | Ni_{0.95}Sn_{0.05}O | 19.5 | 65.0 | 12.7 | 28.5 | 67.0 | 19.1 | 29.5 | 67.0 | 19.8 |
| 17 | Ni_{0.85}Nb_{0.15}O | 10.1 | 54.7 | 5.5 | 19.7 | 57.7 | 11.4 | 23.3 | 58.8 | 13.7 |
| 18 | Ni_{0.85}Nb_{0.15}O | 14.2 | 57.2 | 8.1 | 23.5 | 61.3 | 14.4 | 25.1 | 62.6 | 15.7 |
| 19 | Ni_{0.85}Nb_{0.15}O | 16.3 | 76.2 | 12.4 | 29.5 | 75.3 | 22.2 | 32.5 | 74.8 | 24.3 |

The data in Table 1 and the examples and comparative examples for which data is provided in Table 1 support several observations. First, with the same composition, the catalysts prepared from solid-state synthesis (Ex 1-9) have better activity than those from sol-gel method (CEx B-J). Second, the same examples and comparative examples demonstrate that solid-state synthesis of a catalyst leads to a higher ethylene yield than a catalyst having the same composition, but prepared via sol-gel synthesis. Third, solid-state synthesis is a more straightforward and efficient preparation technique than sol-gel synthesis. Fourth, oxalic acid or an oxalate salt plays an important role in preparing NiO-based catalysts via solid-state synthesis. The NiO and Ni_{0.85}Nb_{0.15}O synthesized without oxalate (CEx A, K) demostrate lower activity and selectivity than those prepared in the presence of oxalate (Ex 1, 10). Fifth, the catalytic performance of NiO catalysts depends upon the nickel precursor used in preparing such catalysts. By way of illustration, Ni_{0.85}Nb_{0.15}O prepared from Ni(NO₃)₂.6H₂O (Ex 10) exhibits much better performance than those prepared from Ni(OH)₂_{_}(Ex 17), Ni(CH₃CO₂)₂ (Ex 18) and NiCl₂_{_}(CEx L, minimal conversion and very low selectivity). Sixth, the optional drying step can be implemented if desired, but is not essential as revealed by a comparison of Ex 19 (no drying step) with Ex 10 (drying step included) which shows similar activity.

### EX 20

### Stability Test on Ni—Ta—O Catalysts

Evaluate catalyst stability (Ex 4 and CEx E by passing a gas mixture of 10% C₂H₆/10% O₂ in helium through the catalytic bed described in the catalyst activity evaluation description held at 330° C., at a total flow rate of 10 mL/min (W/F=0.6 g s/mL), and sample the reaction mixture via on-line gas chromatograph continuously for 50 hours (hr). The results (ethane conversion and ethylene selectivity) are summarized in Table 2.

TABLE 2

| | 5 hr | | 10 hr | | 15 hr | | 20 hr | | 25 hr | |
|---|---|---|---|---|---|---|---|---|---|---|
| | % | % | % | % | % | % | % | % | % | % |
| Catalyst | Conv | Sel. | Conv | Sel | Conv | Sel | Conv | Sel | Conv | Sel |
| Ex-4 | 33.7 | 80.4 | 33.4 | 80.7 | 33.2 | 80.8 | 33.0 | 80.9 | 32.8 | 81.1 |
| CEx E | 20.1 | 80.9 | 19.2 | 81.5 | 18.8 | 81.8 | 18.4 | 82.1 | 18.20 | 82.0 |
| | 30 hr | | 35 hr | | 40 hr | | 45 hr | | 50 hr | |
| | % | % | % | % | % | % | % | % | % | % |
| | Conv | Sel | Conv | Sel | Conv | Sel | Conv | Sel | Conv | Sel |
| Ex-4 | 32.7 | 81.2 | 32.6 | 81.2 | 32.4 | 81.4 | 32.3 | 81.4 | 32.2 | 81.4 |
| CEx E | 18.0 | 82.4 | 17.9 | 82.6 | 17.7 | 82.7 | 17.5 | 82.9 | 17.3 | 82.8 |

From the data presented in Table 2, the catalyst of this invention (Ex 4, solid-state synthesis) when compared to sol-gel synthesized catalysts (CEx E) exhibit not only improved productivity resulting from a combination of higher activity (conversion) and good selectivity but also higher stability with time-on-stream in low temperature (330° C.) ethane ODH.

1. A solvent-free process for synthesizing a nickel oxide-based oxidative dehydrogenation catalyst that comprises sequential steps as follows:
a. mixing without added solvent a combination of a solid nickel precursor, a solid oxalate or oxalic acid and, optionally, a doping amount of a metal precursor for a period of time sufficient to convert the combination to a visually homogenous mixture; and
b. calcining the visually homogeneous mixture at a temperature within a range of from greater than 250° C. to less than 800° C. for a time within a range of from 30 minutes to 360 minutes in an oxygen-containing atmosphere to form a calcined oxidative dehydrogenation catalyst.

2. The process of claim 1, further comprising a sequential intermediate step a′ that follows step a, precedes step b and comprises drying the homogeneous mixture at a temperature within a range of from 50° C. to 90° C. for a period of time within a range of from 10 minutes to 600 minutes to form a dried mixture, the dried mixture thereby replacing the visually homogeneous mixture in step b.

3. The process of claim 1, wherein the solid nickel precursor is selected from a group consisting of nickel nitrate, nickel hydroxide, and nickel acetate and their corresponding hydrated compounds.

4. The process of claim 1, wherein the metal precursor is selected from a group consisting of compounds of Groups IV through VI of the Periodic Table of the Elements, tin, and iron.

5. The process of claim 4, wherein the metal precursor is selected from compounds of a group consisting of tantalum, niobium, titanium, molybdenum, tungsten and zirconium.

6. The process of claim 1, wherein the solid nickel precursor, the oxalate or oxalic acid and, when used, the metal precursor, are present in amounts as follows: from 1 percent by mole (mol %) to 40 mol % solid nickel precursor, from greater than 20 mol % to 98 mol % oxalate or oxalic acid, from greater than or equal to 1 mol % to 40 mol % metal precursor, each mol % being based upon combined moles of solid nickel precursor, oxalate and metal precursor and, in each case, when added together total 100 mol %.

7. The process of claim 6, wherein the solid nickel precursor, the oxalate or oxalic acid and the metal precursor are present in amounts as follows: from 3 mol % to 30 mol %, solid nickel precursor, from 40 mol % to 94 mol %, oxalate or oxalic acid and from 3 mol % to 30 mol % metal precursor, each mol % being based upon combined moles of solid nickel precursor, oxalate or oxalic acid and metal precursor and, in each case, when added together total 100 mol %.

8. A process for effecting oxidative dehydrogenation of ethane using the nickel oxide-based oxidative dehydrogenation catalyst prepared by the process of claim 1, comprising sequential steps as follows:
a. placing the calcined oxidative dehydrogenation catalyst in contact with a feedstream that comprises ethane, oxygen and, optionally, an inert diluent at a temperature of less than 350° C. at a feedstream flow rate within a range of from 50 hr⁻¹ to 10000 hr⁻¹ to yield a product stream that comprises ethylene, carbon dioxide and unreacted ethane.
b. recovering ethylene from the product stream.
